(19) United States
(12) Patent Application Publication
(10) Pub. No.: US 2024/0192201 A1
(43) Pub. Date: Jun. 13, 2024

(54) BRAIN NATRIURETIC PEPTIDE APTAMER FLUORESCENCE DETECTION DEVICE BASED ON SMART PHONE AND SENSING METHOD OF SAME

(71) Applicant: TianJin University, Tianjin (CN)

(72) Inventors: Shuang Li, Tianjin (CN); Ziyue Qin, Tianjin (CN); Dong Ming, Tianjin (CN)

(21) Appl. No.: 18/457,309

(22) Filed: Aug. 28, 2023

Related U.S. Application Data
(63) Continuation of application No. PCT/CN2023/100078, filed on Jun. 14, 2023.

(30) Foreign Application Priority Data
Dec. 12, 2022 (CN) 2022115884141

(51) Int. Cl. G01N 33/53; B01L 3/00; G01N 21/03; G01N 21/64; G01N 33/68

(52) U.S. Cl. CPC G01N 33/53; B01L 3/563; B01L 3/565; G01N 21/03; G01N 21/6486; G01N 33/68; B01L 2300/043; B01L 2300/0663; B01L 2300/0681; G01N 2333/58

(57) ABSTRACT

The present invention discloses a BNP aptamer fluorescence detection device based on a smart phone and a sensing method thereof, including a detection control mechanism, a fluorescence rapid detection mechanism and an aptamer fluorescence sensor. The aptamer fluorescence sensor uses oligonucleotides marked by carboxy fluorescein as an aptamer to capture the BNP specificity. The detection control mechanism receives a fluorescence signal to display test results. The fluorescence rapid detection mechanism uses the OTG function as a plug-in power supply. The present invention fills the gap of portable fluorescence detection for digital BNP.

### SEQUENCE LISTING

This application contains a Sequence Listing XML submitted under the provisions of 37 CFR 1.831(a) and herein incorporated by reference. The Sequence Listing XML includes, in XML format, File Name “SL.xml”; Creation Date: Dec. 3, 2023; Size in bytes: 4,096

### TECHNICAL FIELD

The present invention pertains to the technical field of biosensing, in particular relates to a fluorescence detection device for detecting biomarkers and a sensing method thereof.

### BACKGROUND

At present, a method for determining the brain natriuretic peptide (BNP) concentration in blood includes enzyme-linked immunosorbent assay (ELISA), chemiluminescent immunoassay (CLIA) or the like, but it still has its limitations such as expensiveness, complexness and low sensitivity. ELISA has good specificity, but its sensitivity is poor and its accuracy is quite low. The commercial detection platform for CLIA has high sensitivity and provides a wide detection range, but its costs are often high and its anti-interference ability relatively weakens, limiting its promotion and application. The above methods are of immunoassay by means of specificity identification of antigens and antibodies, and the existence of BNP in various forms in blood often leads to cross-reaction, resulting in overestimation to the BNP concentration.

Therefore, biosensors and immunosensors are becoming a trend in the field of detection, as a tool capable of more accurately and rapidly determining analytes. Accordingly, there is a good prospect in the development of an economic, rapid and effective, highly sensitive and well-selective BNP detection method. A point-of-care testing has gradually risen in response to the development of smart phone, biochemical analysis, aptamer probe sensing technology and other new technologies, and more and more detection devices have developed in pursuit of advantages such as rapidity, portability and efficiency. Although these commercial detection platforms can provide cost-effective mobile healthcare and personalized medical services, commercial technologies applied to clinical applications still face some challenges and unsolved problems, as technological development differs in level. References are as follows.
[1] Dahiya T, Yadav S, Yadav N, et al. Monitoring of BNP cardiac biomarker with major emphasis on biosensing methods: A review[J]. Sensors International, 2021, 2, 100103.
[2] Ashist S K, Luppa P B, Yeo L Y, et al. Emerging Technologies for Next-Generation Point-of-Care Testing[J]. Trends in Biotechnology, 2015, 33 (11): 692-705.

### SUMMARY

In response to the limitations of the above traditional BNP detection device and the needs for the point-of-care testing, the present invention provides a BNP aptamer fluorescence detection device based on a smart phone and a sensing method thereof, which adopts direct plug-in power supply from a smart phone to control detection, and uses a fluorescence rapid detection mechanism as a carrier to integrate an aptamer fluorescence sensor and a detection control mechanism, and finally realize rapid and accurate BNP detection.

In order to solve the above technical problem, the technical scheme adopted in the present invention is as follows:

According to one aspect of the present invention, a BNP aptamer fluorescence detection device based on a smart phone is provided, comprising a detection control mechanism, a fluorescence rapid detection mechanism and an aptamer fluorescence sensor;
the detection control mechanism includes a power supply module, a microcontroller, an excitation light source, a signal acquisition and photoelectric conversion module, a signal chain processing module and a Bluetooth module, the input end of the power supply module is configured to connect with the USB interface of a smart phone, which outputs a power supply voltage to the power supply module, and supplies power to the the detection control mechanism; the microcontroller is connected with the excitation light source, the signal acquisition and photoelectric conversion module and the signal chain processing module by way of signals, and communicate with smart phone through Bluetooth modules; the excitation light source is configured to turn on and off according to the control signal of the microcontroller, and generate excitation light with a set wavelength; the signal acquisition and photoelectric conversion module is configured to capture a fluorescence signal generated form the solution to be detected according to the control signal of the microcontroller, and convert the fluorescence signal into an electrical signal, which is transmitted to the microcontroller; the signal chain processing module is configured to receive electrical signals transmitted by the microcontroller, perform analog-to-digital conversion and filter amplification on the electrical signal, and transmit processed results to the microcontroller; the Bluetooth module is configured to transmit a command signal of a smart phone to the microcontroller, and transmit processed results obtained by the microcontroller to a smart phone to display;

the fluorescence rapid detection mechanism is configured to place the aptamer fluorescence sensor to detect BNP under the control of the detection control mechanism; the fluorescence rapid detection mechanism includes a main box and a smart phone support, the smart phone support is configured to place a smart phone to carry out power supply, detection operation and result display by means of the smart phone; the inside of the main box is provided with an inner shell, which is distanced from the main box to a certain extent; the signal acquisition and photoelectric conversion module and a main control circuit board are fixed and installed on the inner shell, which is provided with a fixing hole of excitation light sources; the main control board integrates the power supply module, the microcontroller, the signal chain processing module and the Bluetooth module; a fluorescent signal reaction table is arranged inside the inner shell, the portion of the fluorescence signal reaction table far away from the fixing hole of excitation light sources is provided with a fluorescence signal reaction cell, which is used to place a quartz cuvette containing the solution to be detected; the portion of the fluorescence signal reaction table close to the fixing hole of excitation light sources is provided with a thread fixing hole for excitation light, which communicates with the fluorescence signal reaction cell, and is coaxial with the fixing hole of excitation light sources, so that the fixing hole of excitation light sources and the thread fixing hole for excitation light jointly fix a LED excitation light source; the connection of the thread fixing hole for excitation light and the fluorescence signal reaction cell is provided with an excitation filter groove, which is used to accommodate an excitation filter; a connection body is set between the fluorescence signal reaction table and the inner shell, and an emitted-light receiving hole is opened inside the connection body; the emitted-light receiving hole communicates with the fluorescence signal reaction cell, and the emitted-light receiving hole and the thread fixing hole for excitation light are vertical with each other in the horizontal direction at the fluorescence signal reaction cell; the connection between the emitted-light receiving hole and the fluorescence signal reaction cell is provided with an emission filter slot, which is used to accommodate a emission filter; thus, the excitation light with a preset wavelength generated from the excitation light source irradiates the fluorescence signal reaction cell via the excitation filter, and the solution to be detected in the quartz cuvette inside the fluorescence signal reaction cell generates a fluorescent signal, which is transmitted to the signal acquisition and photoelectric conversion module through the emitted-light receiving hole after passing through the emission filter;
the aptamer fluorescence sensor is prepared by using oligonucleotides marked by carboxy fluorescein as an aptamer and carboxylated graphene oxide as a fluorescence quencher for the BNP detection, so as to obtain the signal response relation between the BNP concentration and fluorescence intensity according to the analysis of multi-groups of experimental data.

Further, the output end of the power supply module is configured to connect with the microcontroller, the excitation light source and the signal acquisition and photoelectric conversion module, to output a 3.3V supply voltage to the microcontroller, output a 5V supply voltage to the excitation light source and output a ±5V supply voltage to the signal acquisition and photoelectric conversion module, respectively, and the signal chain processing module and the Bluetooth module are powered under a 3.3V voltage output by the microcontroller.

Further, the fluorescence rapid detection mechanism is made from a black ABS resin by integrated molding.

Further, the top surface of the main box is provided with an opening, at which a light-tight flap is arranged; the rear side of the light-tight flap is hinged with the main box, so as to facilitate the replacement of the solution to be detected during a detection operation; the front, back, and side of the main box are provided with openings, at which a light-tight baffle is installed; the light-tight baffle is inserted and removed by means of a baffle slide set on the main box.

Further, the top of the main box is provided with a signal lamp placing hole used to determine whether the main control circuit board functions to power a signal lamp; a wiring port is arranged below the back of the main box, used for the lines led from the inside of the main box.

Further, the front plate, rear plate and side plate define the inner shell, the signal acquisition and photoelectric conversion module is fixed on the surface of the front plate by screws, the main control circuit board is embedded on the surface of the back plate, and the fixing hole of excitation light sources is opened in the middle position of the side plate.

Further, the emitted-light receiving hole is set as a conical hole, the diameter of which gradually shrinks from the fluorescence signal reaction cell to the inner shell, achieving the aggregation of light.

Further, the smart phone support has a slope, a semicircular arc-shaped baffle with an opening is set at the bottom of the slope, and the opening is in correspondence with the position of the USB interface of a smart phone.

Further, the aptamer can capture the BNP specificity and recover fluorescence, the higher the BNP concentration, the greater the fluorescence intensity, and the base sequence of the used oligonucleotides aptamer is as follows:
5′-FAM-TTTTTTTATACGGGAGCCAACACCACCTCTCACATTATATTGTGAAT ACTTCGTGCTGTTTAGAGCAGGTGTGACGGAT-3′.

According to another aspect of the present invention, a sensing method of the BNP aptamer fluorescence detection device based on a smart phone is provided, comprising the steps of:
(1) mixing 100 nmol/L of aptamer solution with 40 μg/ml of carboxylated graphene oxide dispersion liquid by equal volume, and controlling the pH value of the solution at 7.2-7.4, and then leaving it at room temperature for about 20±5 min;
(2) adding the solution to be detected containing a certain BNP concentration into the mixed solution obtained in step (1), then leaving it at room temperature for 35±5 min; and
(3) taking out an appropriate amount of solution to be detected in a quartz cuvette, then placing the quartz cuvette in the fluorescence signal reaction cell, next setting the excitation wavelength as 492 nm, the emission wavelength as 519 nm, and the emission spectrum detection range as 505-610 nm on the smart phone; detecting the fluorescence intensity of the mixed solution, and obtaining the BNP concentration in the solution according to a linear response relation between substance concentration and fluorescence intensity.

The present invention has the following beneficial effects.
(1) The present invention adapted to the common operating habits of people and designed as a portable fluorescence rapid detection structure integrates the aptamer fluorescence sensor and the detection control mechanism, reduces the space volume of the entire device, controls the detection process and signal acquisition in combination with the microcontroller, sensitively captures weak fluorescence signals, carries out photoelectric conversion in combination with the photoelectric conversion module, and enables real-time detection to BNP.
(2) The present invention uses the OTG function of a smart phone for direct plug-in power supply, so that the USB interface being used to output a 5V voltage, not only simplifies wiring operation, but also enables the smart phone to act as mobile power supply to break through the use environment of the detection device, and easy to carry, meeting the requirement of immediate detection.
(3) The present invention uses oligonucleotides as an aptamer for BNP specificity identification, the oligonucleotides are simple in base sequence, easy to obtain, and has low costs, and the aptamer is not easy to bind to other protein antigens, avoids cross-reaction between peptides, has excellent characteristics of high specificity, rapid response and short detection process, and is suitable for rapid detection. Carboxylated graphene oxide acting as a fluorescence quencher has stable chemical properties, low cost, and good ripeness in preparation technology, facilitating the development of low-cost fluorescence sensors.
(4) The present invention detects BNP without complex pre-treatment of the sample to be detected, saving time and costs, and has the advantages such as rapidity and high-efficiency, simple operation, low costs and high sensitivity.

### DESCRIPTION OF DRAWINGS

FIG. 1 is a system diagram of the BNP aptamer fluorescence detection device according to the embodiments of the present invention.

FIG. 2 is an axial assembly diagram of the BNP aptamer fluorescence detection device according to the embodiments of the present invention.

FIG. 3 is a top view and its A-A, B-B section views of the BNP aptamer fluorescence detection device according to the embodiments of the present invention.

FIG. 4 is an axial view of the main box and the smart phone support of the BNP aptamer fluorescence detection device according to the embodiments of the present invention.

FIG. 5 is a fluorescence spectrum of a series of carboxylated graphene oxide solutions and the solution only containing 100 nmol/L of aptamers.

FIG. 6 is a fluorescence spectrum of a series of carboxylated graphene oxide solutions and the mixed solution containing 100 nmol/L of aptamers and 1000 pg/ml of BNP.

FIG. 7 is a bar comparison diagram of the peak and peak difference under the conditions of FIG. 5 and FIG. 6.

FIG. 8 shows the optimal detection time of fluorescence quenching.

FIG. 9 shows the optimal detection time of fluorescence recovery.

FIG. 10 is a fluorescence spectrum of BNP solution with a series of concentration gradients detected the aptamer fluorescence sensor.

FIG. 11 shows a linear response curve of the detected concentration and fluorescence intensity of BNP solution.
Where, 1—detection control mechanism; 101—power supply module; 102—microcontroller; 103—excitation light source; 104—signal acquisition and photoelectric conversion module; 105—signal chain processing module; 106—Bluetooth module; 2—fluorescence rapid detection mechanism; 201—main box; 202—smart phone support; 203—light-tight flap; 204—signal lamp placing hole; 205—light-tight baffle; 206—baffle slide; 207—wiring port; 208—inner shell; 209—fixing hole; 210—fixing hole for the excitation light source; 211—main control circuit; 212—fluorescent signal reaction table; 213—base; 214—fluorescence signal reaction cell; 215—thread fixing hole for excitation light; 216—excitation filter groove; 217—emitted-light receiving hole; 218—emission filter slot; 3—aptamer fluorescence sensor.

### DETAILED DESCRIPTION

To further understand the content, characteristics and effects of the present invention, we shall make descriptions in detail in combination with the embodiments and drawings as follows.

The present invention stems from fluorescence sensing technology of aptamers, using carboxylated graphene oxide as a fluorescence resonance energy transfer platform used for quenching fluorescence on the aptamers marked by carboxy fluorescein. With addition of the BNP, the aptamer achieves the BNP specificity identification so that the aptamer is separated from the graphene surface to recover the fluorescence; therefore based on this principle, the BNP concentration in solution can be accurately determined. In order to meet the requirements of immediate detection, the fluorescence rapid detection mechanism 2 is designed as a carrier, powered by a smart phone, and the detection control mechanism 1 is configured to collect, process, transmit data, and finally transmit data to the smart phone through Bluetooth to display the test results.

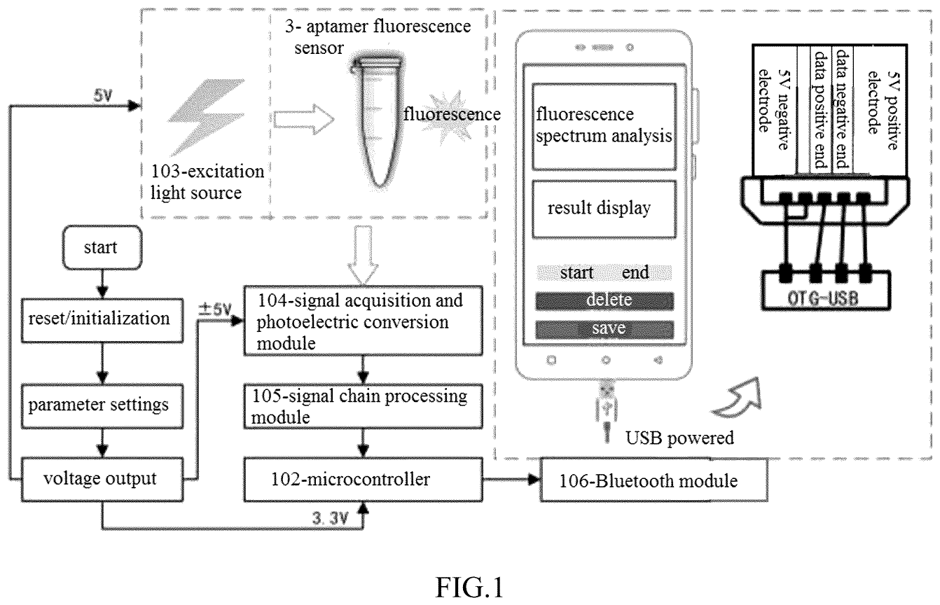

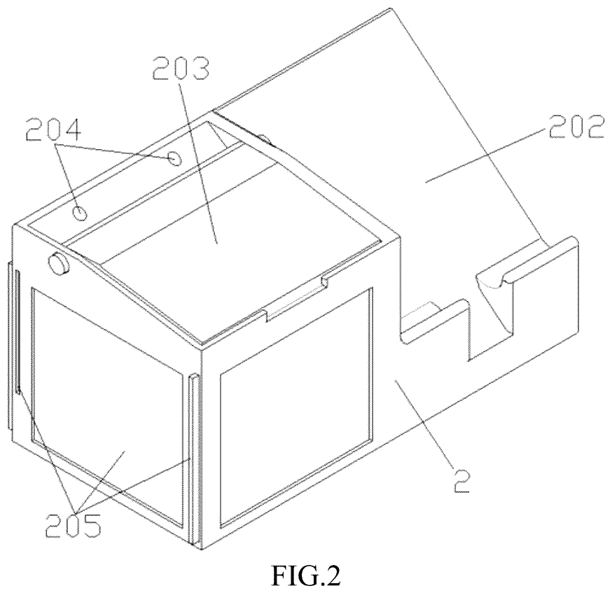

As shown in FIG. 1, the present embodiment provides a BNP aptamer fluorescence detection device based on a smart phone, which includes the detection control mechanism 1, the fluorescence rapid detection mechanism 2 and the aptamer fluorescence sensor 3.

The detection control mechanism 1 is configured to regulate the integrated detection process, achieve fluorescence signal acquisition and photoelectric conversion, data processing and output and other processes, including the power supply module 101, the microcontroller 102, the excitation light source 103, the signal acquisition and photoelectric conversion module 104, the signal chain processing module 105 and the Bluetooth module 106.

The input end of the power supply module 101 is configured to connect with the USB interface of a smart phone, which outputs a 5V power supply voltage by means of its OTG function, and supplies power to the entire detection control mechanism 1. The output end of the power supply module 101 is configured to connect with the microcontroller 102 and the signal acquisition and photoelectric conversion module 104, to output a 3.3V supply voltage to the microcontroller 102 and output a ±5V supply voltage to the signal acquisition and photoelectric conversion module 104, respectively. The excitation light source 103 is directly powered under a 5V voltage output by the power supply module 101, and the signal chain processing module 105 and the Bluetooth module 106 are powered under a 3.3V voltage output by the power supply module 101.

Furthermore, based on the direct plug-in power supply mode of the OTG function of the smart phone, the wiring principle for the USB interface is shown in FIG. 1, in which the ground electrode and the dead end on the USB circuit interface are used as the negative electrode of the 5V power supply, and the power line is used as the positive electrode of the 5V power supply. This configuration achieves the 5V voltage output of the smart phone. which is used as a mobile power supply to supply power to the detection control mechanism 1, enriching use environments and conforming to the development of immediate detection technology.

The microcontroller 102 is connected with the excitation light source 103, the signal acquisition and photoelectric conversion module 104 and the signal chain processing module 105 by way of signals, and can be connected to the smart phone by way of signals via the Bluetooth module 106. For example, the microcontroller 102 may be an STM32F103C8T6 chip. The microcontroller 102 is configured to control the excitation light source 103 on and off, and control the excitation light source 103 to emit excitation light with a preset wavelength to irradiate the solution to be detected in the quartz cuvette; to control the signal acquisition and photoelectric conversion module 104 to capture and convert fluorescence signals, and receive electrical signals output form the signal acquisition and photoelectric conversion module 104; to transmit electrical signals to the signal chain processing module 105, and form processed results according to the information processed by the signal chain processing module 105; and to receive command signals from the smart phone via the Bluetooth module 106, and transmit processed information to the smart phone to display the test results.

The excitation light source 103 is configured to turn on and off according to the control signal of the microcontroller 102, and generate excitation light with a set wavelength according to the control signal of the microcontroller 102, the excitation light irradiates the solution to be detected in the quartz, cuvette via an excitation optical filter, enabling the solution to be detected in the quartz cuvette to generate a fluorescence signal. For example, an LED light source with a wavelength of 492 nm may be used as the excitation light source 103.

The signal acquisition and photoelectric conversion module 104 is configured to capture a fluorescence signal generated form the solution to be detected in the quartz cuvette, and convert the fluorescence signal into an electrical signal, which is transmitted to the microcontroller 102. For example, the signal acquisition and photoelectric conversion module 104 may be in the form of a silicon photomultiplier tube module (C13365-3050SA), the supply voltage of which is ±5V, and output through the ±5V conversion circuit in the power supply module 101, and which can sensitively capture the fluorescence signal and convert it into a voltage signal to be output.

The signal chain processing module is configured to receive electrical signals transmitted by the microcontroller 102, perform analog-to-digital conversion and filter amplification on the electrical signal, and transmit processed results to the microcontroller 102.

The Bluetooth module 106 is configured to transmit a command signal of a smart phone to the microcontroller 102, and transmit processed results obtained by the microcontroller 102 to a smart phone to display test results.

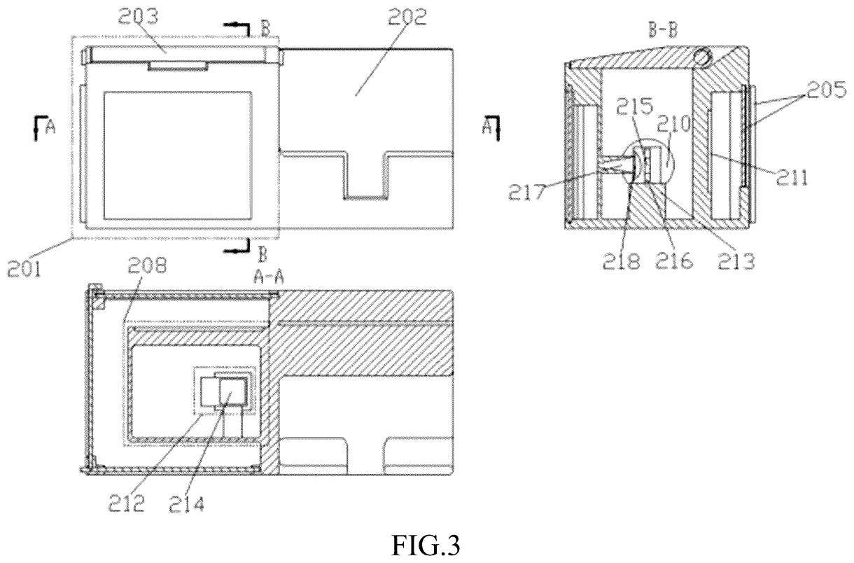

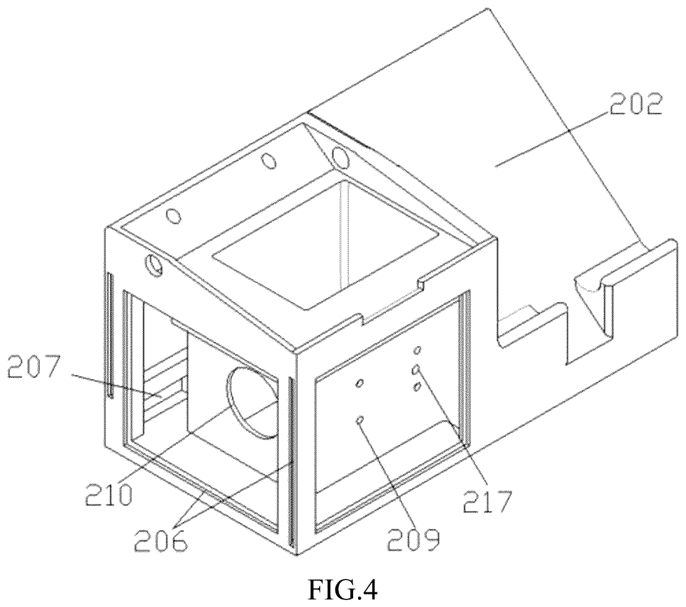

As shown in FIG. 2-4, the fluorescence rapid detection mechanism 2 is designed by way of 3D modeling software SOLIDWORKS based on the ergonomics principle, having a total volume of not more than 20 cm×10 cm×10 cm, and an appearance mainly divided into two parts: the main box 201 and the smart phone support 202. Preferably, the main box 201 and the smart phone support 202 are integrated. The main box 201 is configured to place the aptamer fluorescence sensor 3 to detect the BNP under the control of the detection control mechanism 1, and the smart phone support 202 is configured to place the smart phone for power supply, detection operation, result display and other functions.

Preferably, the fluorescence rapid detection mechanism 2 adopts a light-tight black ABS resin as a 3D printing material, which makes free with the detection environment of the aptamer fluorescence sensor and ensures the smooth progress of integrated immediate BNP detection.

The main box 201 is generally in the form of a square shell, and its top, front, back, and side are provided with square openings to facilitate the installation and removal of various parts in the detection and control mechanism 1.

The square opening on the top of the main box 201 is provided with the light-tight flap 203, and the rear side of the light-tight flap 203 is connected to the main box 201 by means of a pin shaft, thereby forming an articulation with the main box 201. The light-tight flap 203 shades the opening of the top surface of the main box 201, and facilitates the replacement of the solution to be detected during the detection operation. Preferably, the main box 201 is symmetrically provided with two signal lamp placing holes 204 behind the light-tight flap 203, the signal lamp placing hole 204 has an aperture size to be designed to fit a common light-emitting diode, and used for installing a signal lamp to determine whether the main control circuit board is powered normally.

The square openings of the front, rear and side of the main box 201 are provided with the light-tight baffles 205, respectively, which are used to open, close and shade the front, rear and side of the main box 201. The main box 201 is provided with the baffle slide 206, in correspondence with each light-tight baffle 205, thus three baffle slides 206 are used to insert the light-tight baffle 205, respectively. Generally, the three light-tight baffles 205 are installed sequentially on the back, side, front of the main box 201, and disassembled sequentially from the front, side, and back of the main box 201. Accordingly, the rear light-tight baffle 205 is provided with a slot for plugging in the side light-tight baffle 205, and the side light-tight baffle 205 is also provided with a slot for plugging in the front light-tight baffle 205.

Preferably, the wiring port 207 is arranged below the square opening behind the main box 201, facilitating the sorting and external connection of the lines led from the inside of the main box 201.

The inside of the main box 201 is integrally connected with the inner shell 208, and the front plate, rear plate and side plate fixed on the bottom plate of the main box 201 define the inner shell 208, and the front plate, back plate and side plate are distanced from the front, back and side of the main box 201 to a certain extent, respectively.

The front plate of the inner shell 208 is provided with the fixing hole 209 of the signal acquisition and photoelectric conversion module, the fixing hole 209 of the signal acquisition and photoelectric conversion module has a size to be designed to fit the signal acquisition and photoelectric conversion module 104, and includes four through-holes in correspondence with the four corners of the signal acquisition and photoelectric conversion module 104, respectively, enabling the signal acquisition and photoelectric conversion module 104 to be attached on the surface of the front plate of the inner shell 208 and to be fixed thereon.

The side plate of the inner shell 208 is provided with a circular through-hole acting as the fixing hole 210 for the excitation light source.

The outer side of the rear plate of the inner shell 208 is provided with a rectangular groove used to fix the main control circuit board. The main control board integrates the power supply module 101 of the sensing control mechanism 1, the microcontroller 102, the signal chain processing module 105 and the Bluetooth module 106.

The fluorescent signal reaction table 212 having a cuboid profile is arranged inside the inner shell 208. The fluorescence signal reaction table 212 is fixed on the surface of the bottom plate of the main box 201 by means of the base 213, so that the fluorescence signal reaction table 212 has a given height.

The portion of the fluorescence signal reaction table 212 far away from the fixing hole 210 of excitation light sources is provided with a groove with an upward opening, which acts as a fluorescence signal reaction cell 214. The fluorescence signal reaction cell 214 is used to place a quartz cuvette containing the solution to be detected.

The portion of the fluorescence signal reaction table 212 close to the fixing hole 210 of excitation light sources is provided with an internal threaded hole, which acts as the thread fixing hole 215 for excitation light. The thread fixing hole 215 for excitation light communicates with the fluorescence signal reaction cell 214, and is coaxial with the fixing hole 210 of excitation light sources. The fixing hole 210 of excitation light sources and the thread fixing hole 215 for excitation light jointly fix the LED excitation light source, which is used to generate and transmit excitation light in the horizontal direction.

The connection of the thread fixing hole 215 for excitation light and the fluorescence signal reaction cell 214 is provided with the excitation filter groove 216, which is used to accommodate the excitation filter, so as to achieve the purpose of filtering excitation stray light and improving the excitation efficiency.

A connection body is set between the fluorescence signal reaction table 212 and the front plate of the inner shell 208, and the emitted-light receiving hole 217 is opened inside the connection body. The emitted-light receiving hole 217 communicates with the fluorescence signal reaction cell 214, and forms a through hole on the front plate of the inner shell 208, the emitted-light receiving hole 217 and the thread fixing hole 215 for excitation light are vertical with each other in the horizontal direction at the fluorescence signal reaction cell 214. Thus, the excitation light with a preset wavelength generated from the excitation light source 103 irradiates the fluorescence signal reaction cell 214 via the excitation filter, and the solution to be detected in the quartz cuvette inside the fluorescence signal reaction cell 214 generates a fluorescent signal, and the fluorescent emitted light transmits at an angle of 90° with respect to the excitation light. Since the generated fluorescent signal is relatively weak, the emitted-light receiving hole 217 is preferably set as a conical hole, which gradually shrinks from the fluorescence signal reaction cell 214 to the front plate of the inner shell 208, achieving the aggregation of light and finally transmitting it to the signal acquisition and photoelectric conversion module 104. In addition, the inner wall of the emitted-light receiving hole 217 can also be painted to improve the light reflection efficiency and more conduce the aggregation of fluorescent emitted light.

The connection between the emitted-light receiving hole 217 and the fluorescence signal reaction cell 214 is provided with the emission filter slot 218, which is used to accommodate an emission filter. Preferably, the emission filter and the excitation filter both adopt a high-transmittance visible light filter with a wavelength range of 400 nm-700 nm, which is used to screen optical wavelengths.

As a preferred embodiment, the main body of a smart phone support 202 has a slope, and the slope angle is suitable to the human's view range, for erecting a smart phone. A semicircular arc-shaped baffle with an opening is set at the bottom of the slope, and the opening is in correspondence with the position of the USB interface of a smart phone, and convenient for plugging and unplugging form the USB interface and power supply. Thus, the smart phone support 202 is used to place a smart phone, convenient for power supply, detection operation and result display, playing an important role on the structured design of the fluorescence rapid detection mechanism and the realization of the integrated fluorescence rapid BNP detection.

The aptamer fluorescence sensor 3 detects the fluorescence signal generated by the solution to be detected in the quartz cuvette, and rapidly determines the BNP concentration based on fluorescence sensing technology. The oligonucleotides marked by carboxy fluorescein are used as an aptamer to capture the BNP specificity. Based on the principle of fluorescence resonance energy transfer, carboxylated graphene oxide is used as a fluorescence quenching agent, and with addition of the BNP, the aptamer achieves the BNP specificity identification so that the aptamer recovers the fluorescence. The fluorescence intensity is determined by using an UV-visible fluorescence spectrometer to obtain multi-groups of fluorescence spectra, and the signal response relation between substance concentration and fluorescence intensity is obtained according to the analysis of multi-groups of experimental data.

Among them, the base sequence of the aptamer marked by carboxy fluorescein is as follows:

5′-FAM-TTTTTTTATACGGGAGCCAACACCACCTCTCACATTATATTG
TGAATACTTCGTGCTGTTTAGAGCAGGTGTGACGGAT-3′.

The surface of carboxylated graphene oxide has a large number of hydrophilic functional groups, which have wettability, surface activity and biological properties to a certain extent; the base sequence of this aptamer has two BNP specificity identification sites, and an Oligo (dT) thymine sequence is added between the group marked by fluorescence and the true DNA sequence to increase the steric hindrance therebetween and prevent the reaction therebetween, enabling more stable aptamers, and higher specificity identification.

Considering the effects of BNP and carboxylated graphene oxide on an aptamer fluorescence signal, the present invention also designs a pre-experiment to determine the optimal experimental conditions for fluorescence BNP detection, such as the optimal concentration of carboxylated graphene oxide solution, the optimal detection time for fluorescence quenching, and the optimal detection time for fluorescence recovery. The specific experimental steps are as follows:

(1) Preparation for reagents and materials

100 nmol/L of aptamer solution: select an appropriate amount of high-concentration aptamer solution, then heat it in a 95° C. water bath for 5 min, next cool it naturally to room temperature and dilute it; 1000 pg/ml of BNP solution; a series of solutions to be detected containing BNP; a series of carboxylated graphene oxide solutions, an UV-visible fluorescence spectrometer, etc.

(2) Experiment on the optimal concentration of carboxylated graphene oxide

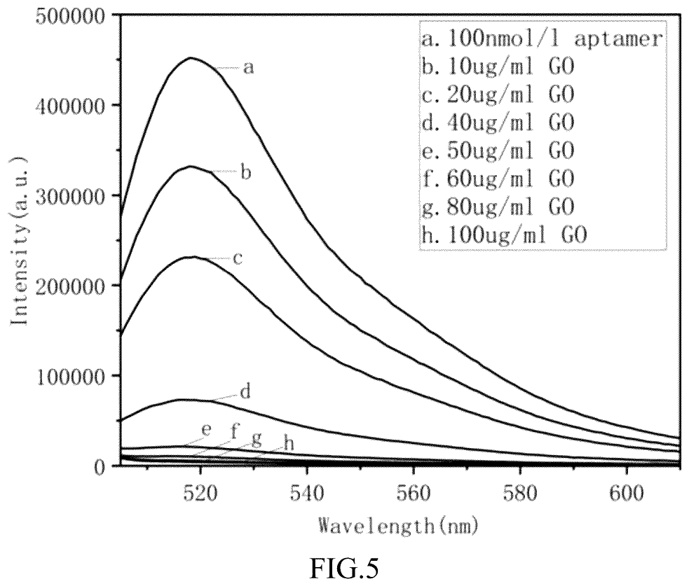

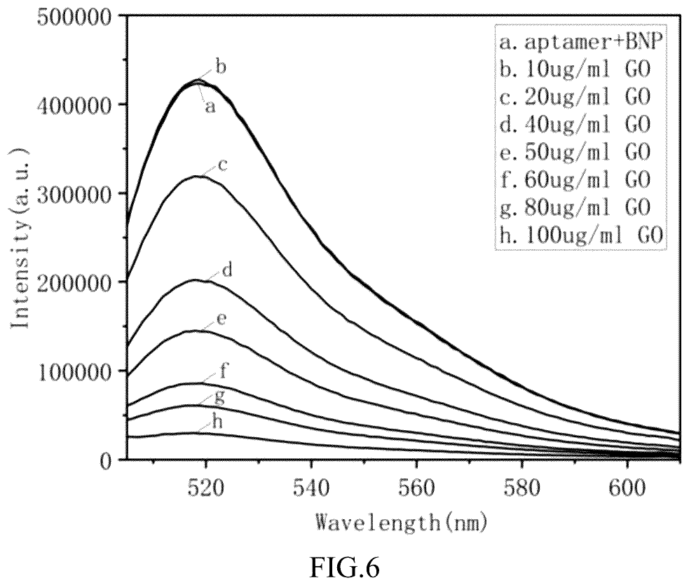

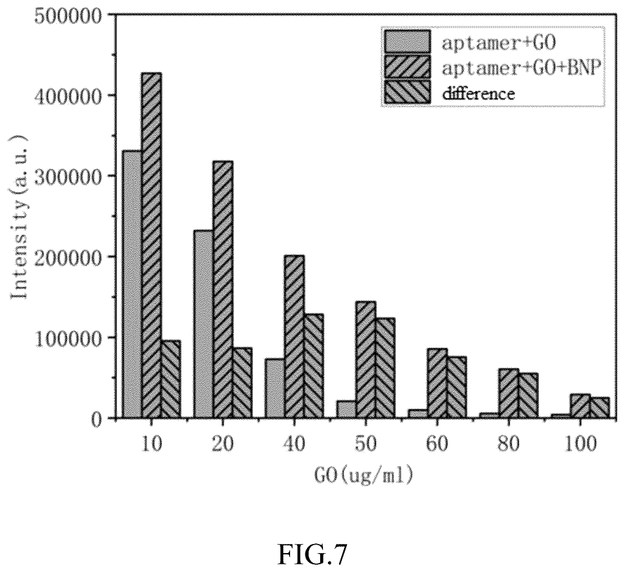

A series of carboxylated graphene oxide solutions is mixed with the solution only containing 100 nmol/L of aptamers and the mixed solution containing 100 nmol/L of aptamers and 1000 pg/ml of BNP, respectively. To ensure that other conditions remain unchanged, the fluorescence intensity detection is carried out by using an UV-visible fluorescence spectrometer to obtain the fluorescence intensity values at different concentrations of carboxylated graphene oxide. Comparison is made on the peak value and the peak difference under the two conditions to obtain the optimal concentration value of carboxylated graphene oxide. The experimental results are shown in FIGS. 5-7, and the optimal concentration of carboxylated graphene oxide is 40 ug/ml.

(3) Optimal detection time

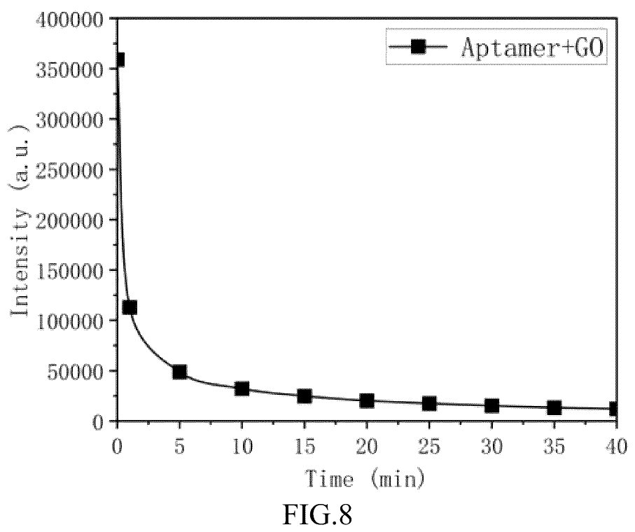

The optimal detection time for fluorescence quenching: mix 100 nmol/L of aptamer solution with 40 ug/ml of carboxylated graphene oxide solution by equal volume, and control the pH value of the solution at 7.2-7.4, and then immediately take out an appropriate amount of mixed solution for fluorescence detection, which is recorded as 0 min, next perform detection every 5 min, which is recorded as 0 min, 5 min, 10 min, etc. Comparison is made on the fluorescence intensity peak values for each detection to estimate the optimal detection time of fluorescence quenching. As shown in FIG. 8, the optimal detection time of fluorescence quenching is 20±5 min.

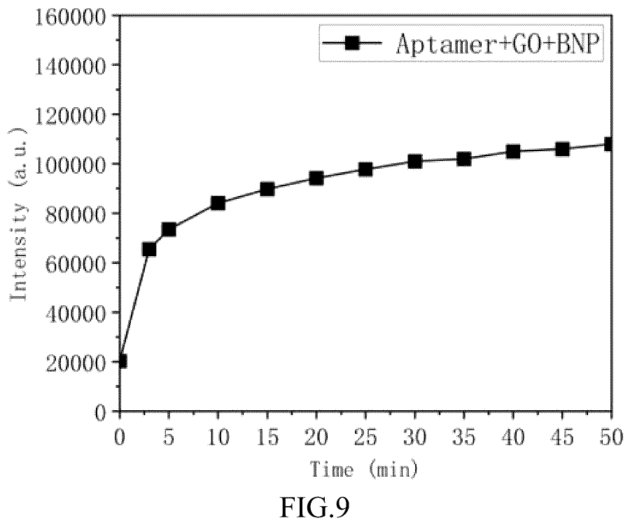

The optimal detection time for fluorescence recovery: mix 100 nmol/L of aptamer solution with 40 ug/ml of carboxylated graphene oxide solution by equal volume, and control the pH value of the solution at 7.2-7.4, and then leave it at room temperature for about 20 min. Then, add 1000 pg/ml of BNP solution to mix, immediately take out an appropriate amount of mixed solution for fluorescence detection, which is recorded as 0 min, next perform detection every 5 min, which is recorded as 0) min, 5 min, 10 min, etc. Comparison is made on the fluorescence intensity peak values for each detection, comparison is made on the fluorescence intensity at different time, to estimate the optimal detection time of fluorescence recovery. As shown in FIG. 9, the optimal detection time of fluorescence recovery is 35±5 min.

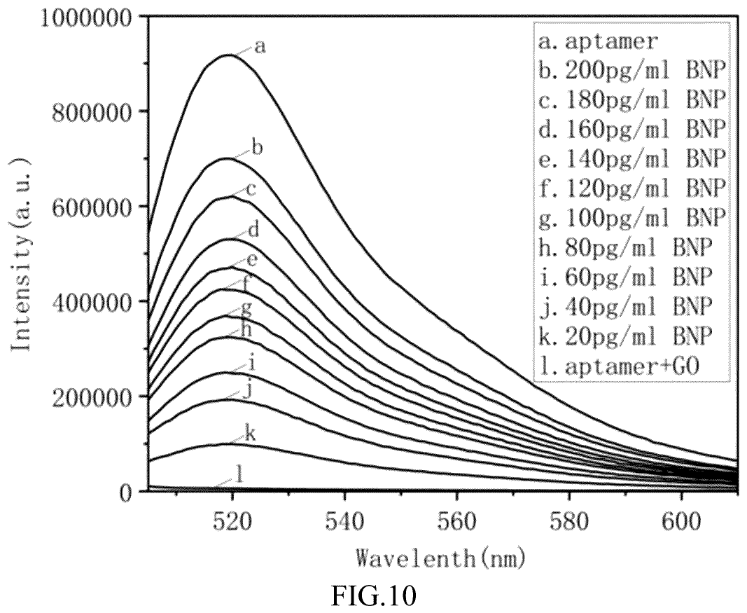

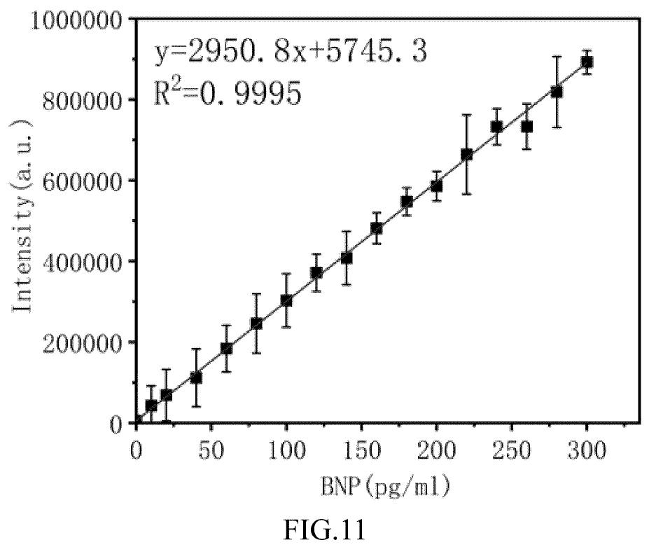

The signal response relationship between BNP concentration and fluorescence intensity obtained by detecting BNP in the present invention, its specific detection steps are as follows:
(1) Blank control experiment: individually detect the fluorescence intensity of 100 nmol/L aptamer solution, and set the excitation wavelength as 492 nm, the emission wavelength as 519 nm, and the emission spectrum detection range as 505-610 nm, then use the obtained results as controls.
(2) Mix 100 nmol/L of aptamer solution with 40 ug/ml of carboxylated graphene oxide solution by equal volume, and control the pH value of the solution at 7.2-7.4, and then leave it at room temperature for about 20 min. Then add BNP solution having a series of concentration gradients, respectively, leave it at room temperature for about 35 min, next take out an appropriate amount of mixed solution for fluorescence detection, and record fluorescence spectra at different concentrations. As shown in FIG. 10, if the aptamer solution is alone detected, it has the highest fluorescence intensity, and if the BNP concentration solution is 0) and carboxylated graphene oxide is used as a quencher, the solution has the lowest fluorescence intensity. With the increase of the BNP concentration, the fluorescence intensity of the solution gradually increases.
(3) After sorting out the above experimental results and process data, we obtain the linear response equation of BNP concentration and fluorescence intensity as y=2950.8x+5745.3 (R2=0.9995), and the linear fitting curve is shown in FIG. 11.

In summary, the present invention adopters a BNP aptamer fluorescent sensing method based on a smart phone, its specific detection steps are as follows:
(1) Actuating your smart phone and powering on, observing the power signal lamp, judging whether the detection circuit functions, checking whether the fluorescence rapid detection mechanism 2 can functions, and whether it meets the requirements of the light-tight environment.
(2) Placing the quartz cuvette containing the solution to be detected in the fluorescence signal reaction cell 214 in the main box 201, closing the light-tight flap 203, and then setting the excitation wavelength as 492 nm, the emission wavelength as 519 nm, and the emission spectrum detection range as 505-610 nm on the smart phone operation interface, finally actuating the excitation light source 103 for detection, the results of which are displayed on the smart phone.
(3) Completing the detection, saving the data, powering off the smart phone, opening the light-tight flap 203, then taking out the solution to be detected and disposing of it safely.

In summary, the present invention provides a BNP aptamer fluorescence detection device based on a smart phone and a sensing method thereof, by using the carboxylated graphene oxide as a quencher, designing the aptamer to achieve the BNP specificity identification, creating the aptamer fluorescence sensor 3 for the BNP detection, designing the fluorescence detection mechanism 2 which can use direct plug-in mobile power supply of a smart phone, and creating a point-of-care testing mobile medical rapid detection platform in combination with modularization and integration conception, thereby breaking through the limitations of traditional detection methods in fixed use environment and large-scale detection equipment, and having good development prospects in the field of low-cost and high-efficiency rapid BNP detection.

Although we have described the preferred embodiments of the present invention in combination with drawings as above, but the present invention is not limited to the above specific embodiments, which only give typical examples, not constraints on the present invention. A person skilled in the art taught by the present invention, without departing from the protection scope of claims and the essence of inventions, may also make many variations, which fall within the protection scope of the present invention.

1. A BNP aptamer fluorescence detection device based on a smart phone, comprising a detection control mechanism, a fluorescence rapid detection mechanism and an aptamer fluorescence sensor;
wherein the detection control mechanism includes a power supply module, a microcontroller, an excitation light source, a signal acquisition and photoelectric conversion module, a signal chain processing module and a Bluetooth module, the input end of the power supply module is configured to connect with the USB interface of a smart phone, which outputs a power supply voltage to the power supply module, and supplies power to the the detection control mechanism; the microcontroller is connected with the excitation light source, the signal acquisition and photoelectric conversion module and the signal chain processing module by way of signals, and can be connected to the smart phone by way of signals through the Bluetooth module; the excitation light source is configured to turn on and off according to the control signal of the microcontroller, and generate excitation light with a set wavelength; the signal acquisition and photoelectric conversion module is configured to capture a fluorescence signal generated form the solution to be detected according to the control signal of the microcontroller, and convert the fluorescence signal into an electrical signal, which is transmitted to the microcontroller; the signal chain processing module is configured to receive electrical signals transmitted by the microcontroller, perform analog-to-digital conversion and filter amplification on the electrical signal, and transmit processed results to the microcontroller; the Bluetooth module is configured to transmit a command signal of a smart phone to the microcontroller, and transmit processed results obtained by the microcontroller to a smart phone to display;
the fluorescence rapid detection mechanism is configured to place the aptamer fluorescence sensor to detect BNP under the control of the detection control mechanism; the fluorescence rapid detection mechanism includes a main box and a smart phone support, the smart phone support is configured to place a smart phone to carry out power supply, detection operation and result display by means of the smart phone; the inside of the main box is provided with an inner shell, which is distanced from the main box to a certain extent; the signal acquisition and photoelectric conversion module and a main control circuit board are fixed and installed on the inner shell, which is provided with a fixing hole of excitation light sources; the main control board integrates the power supply module, the microcontroller, the signal chain processing module and the Bluetooth module; a fluorescent signal reaction table is arranged inside the inner shell, the portion of the fluorescence signal reaction table far away from the fixing hole of excitation light sources is provided with a fluorescence signal reaction cell, which is used to place a quartz cuvette containing the solution to be detected; the portion of the fluorescence signal reaction table close to the fixing hole of excitation light sources is provided with a thread fixing hole for excitation light, which communicates with the fluorescence signal reaction cell, and is coaxial with the fixing hole of excitation light sources, so that the fixing hole of excitation light sources and the thread fixing hole for excitation light jointly fix a LED excitation light source; the connection of the thread fixing hole for excitation light and the fluorescence signal reaction cell is provided with an excitation filter groove, which is used to accommodate an excitation filter; a connection body is set between the fluorescence signal reaction table and the inner shell, and an emitted-light receiving hole is opened inside the connection body; the emitted-light receiving hole communicates with the fluorescence signal reaction cell, and the emitted-light receiving hole and the thread fixing hole for excitation light are vertical with each other in the horizontal direction at the fluorescence signal reaction cell; the connection between the emitted-light receiving hole and the fluorescence signal reaction cell is provided with an emission filter slot, which is used to accommodate a emission filter; thus, the excitation light with a preset wavelength generated from the excitation light source irradiates the fluorescence signal reaction cell via the excitation filter, and the solution to be detected in the quartz cuvette inside the fluorescence signal reaction cell generates a fluorescent signal, which is transmitted to the signal acquisition and photoelectric conversion module through the emitted-light receiving hole after passing through the emission filter;
the aptamer fluorescence sensor is prepared by using oligonucleotides marked by carboxy fluorescein as an aptamer and carboxylated graphene oxide as a fluorescence quencher for the BNP detection, so as to obtain the signal response relation between the BNP concentration and fluorescence intensity according to the analysis of multi-groups of experimental data.

2. The BNP aptamer fluorescence detection device based on a smart phone according to claim 1, wherein the output end of the power supply module is configured to connect with the microcontroller, the excitation light source and the signal acquisition and photoelectric conversion module, to output a 3.3V supply voltage to the microcontroller, output a 5V supply voltage to the excitation light source and output a ±5V supply voltage to the signal acquisition and photoelectric conversion module, respectively, and the signal chain processing module and the Bluetooth module are powered under a 3.3V voltage output by the microcontroller.

3. The BNP aptamer fluorescence detection device based on a smart phone according to claim 1, wherein the fluorescence rapid detection mechanism is made from a black ABS resin by integrated molding.

4. The BNP aptamer fluorescence detection device based on a smart phone according to claim 1, wherein the top surface of the main box is provided with an opening, at which a light-tight flap is arranged; the rear side of the light-tight flap is hinged with the main box, so as to facilitate the replacement of the solution to be detected during a detection operation; the front, back, and side of the main box are provided with openings, at which a light-tight baffle is installed; the light-tight baffle is inserted and removed by means of a baffle slide set on the main box.

5. The BNP aptamer fluorescence detection device based on a smart phone according to claim 1, wherein the top of the main box is provided with a signal lamp placing hole used to determine whether the main control circuit board functions to power a signal lamp; a wiring port is arranged below the back of the main box, used for the lines led from the inside of the main box.

6. The BNP aptamer fluorescence detection device based on a smart phone according to claim 1, wherein the front plate, rear plate and side plate define the inner shell, the signal acquisition and photoelectric conversion module is fixed on the surface of the front plate by screws, the main control circuit board is embedded on the surface of the back plate, and the fixing hole of excitation light sources is opened in the middle position of the side plate.

7. The BNP aptamer fluorescence detection device based on a smart phone according to claim 1, wherein the emitted-light receiving hole is set as a conical hole, the diameter of which gradually shrinks from the fluorescence signal reaction cell to the inner shell, achieving the aggregation of light.

8. The BNP aptamer fluorescence detection device based on a smart phone according to claim 1, wherein the smart phone support has a slope, a semicircular arc-shaped baffle with an opening is set at the bottom of the slope, and the opening is in correspondence with the position of the USB interface of a smart phone.

9. The BNP aptamer fluorescence detection device based on a smart phone according to claim 1, wherein the aptamer can capture the BNP specificity and recover fluorescence, the higher the BNP concentration, the greater the fluorescence intensity, and the base sequence of the used oligonucleotides aptamer is as follows:
5′-FAM-TTTTTTTATACGGGAGCCAACACCACCTCTCACATTATATTG
TGAATACTTCGTGCTGTTTAGAGCAGGTGTGACGGAT-3′.

10. A sensing method of the BNP aptamer fluorescence detection device based on a smart phone, comprising the steps of:
(1) mixing 100 nmol/L of aptamer solution with 40 μg/ml of carboxylated graphene oxide dispersion liquid by equal volume, and controlling the pH value of the solution at 7.2-7.4, and then leaving it at room temperature for about 20±5 min;
(2) adding the solution to be detected containing a certain BNP concentration into the mixed solution obtained in step (1), then leaving it at room temperature for 35±5 min; and
(3) taking out an appropriate amount of solution to be detected in a quartz cuvette, then placing the quartz cuvette in the fluorescence signal reaction cell, next setting the excitation wavelength as 492 nm, the emission wavelength as 519 nm, and the emission spectrum detection range as 505-610 nm on the smart phone; detecting the fluorescence intensity of the mixed solution, and obtaining the BNP concentration in the solution according to a linear response relation between substance concentration and fluorescence intensity.


SEQUENCE LISTING (1 sequence)

SEQ ID NO: 1 - DNA, 79 bp, synthetic construct
  source 1..79: mol_type = other DNA
tttttttatacgggagccaacaccacctctcacattatattgtgaatacttcgtgctgtttagagcaggtgtgacggat